(19) United States
(12) Patent Application Publication
(10) Pub. No.: US 2018/0155410 A1
(43) Pub. Date: Jun. 7, 2018

(54) THERAPY FOR ENTERIC INFECTIONS

(71) Applicant: Thomas Julius Borody, Sydney (AU)

(72) Inventor: Thomas Julius Borody, Sydney (AU)

(21) Appl. No.: 15/828,260

(22) Filed: Nov. 30, 2017

Related U.S. Application Data
(63) Continuation of application No. 13/497,118, filed on Mar. 27, 2012, now abandoned.
(60) Provisional application No. 61/245,277, filed on Sep. 23, 2009.

(51) Int. Cl. C07K 16/02; C07K 16/12; A61K 35/74; A61K 39/00

(52) U.S. Cl. CPC C07K 16/02; C07K 16/1282; C07K 2317/11; A61K 2039/505; A61K 35/74

(57) ABSTRACT

A method and composition for treating enteric pathogen infections in animals suffering from such infections or displaying diseases or conditions consistent with such infections or for preventing or reducing the likelihood of enteric pathogen infections in animals at risk for developing such infections.

### FIELD OF INVENTION

Non-antibiotic therapy for enteric infections.

### BACKGROUND OF THE INVENTION

The gastrointestinal (“GI”) tract is frequently infected by various pathogens. Some transiently infect the bowel flora and mucosa and are removed by the endogenous bacteria or other immune mechanisms (e.g., colonization resistance). Such infections include various strains of Salmonella, Shigella, Campylobacter and various other entero-viruses. There are other enteric infections, however, which are capable of infecting the gastrointestinal tract chronically and result in mild to devastating symptoms and outcomes. Some examples of these pathogens are Clostridium difficile, Clostridium perfringens, Bacillus cereus, Clostridium botulinum, Clostridum tetani, Clostridium welchii, Clostridium sordelli, and various E. coli strains. Still other enteric pathogens lead to the acute infections, which can be overwhelming. Among these pathogens are vibro cholera, Campylobacter jejuni, and Salmonella typhi.

Clostridial infections of the gastrointestinal tract, for example, may result in a group of clinical ‘phenotypic’ presentations. For example, Clostridium botulinum, may result in several clinical presentations, including fatal botulism due to ingestion of contaminated food, via wound infection, and classically in infant botulism through the colonization of the immature infant flora, between 3 weeks and 11 months after birth, with C botulinum, subsequent toxin production and its entry into the blood with devastating consequences [S Arnon, J Infectious Diseases 1986; 154:201]. Sudden Infant Death Syndrome (SIDS) in a similar fashion may be mediated by C botulinum or a closely related pathogen with its entry into the gastrointestinal tract of infants precipitating cardio-respiratory arrest during sleep [S Arnon et al, Lancet 1978; June 17:1273-76; Peterson et Rev. Infect. Dis 1:630 1979].

Another example of these enteric pathogens and their infections is Clostridium tetani which is generally associated with classic neuronal tetanus. Evidence is accumulating, however, that certain strains of Clostridium tetani which enter young children's gastrointestinal tract, e.g., following antibiotic use, may chronically elaborate neurotoxins that are capable of reaching the central nervous system retrograde via the vagus nerve or by the circulation and that result in a clinical condition called ‘Autism spectrum’ [E. Bolte Medical Hypotheses S1. 133-144; 1998]. This includes Autism, Asbergers and Rhett Syndrome as well as ADD and ADHD.

Clostridium perfringens is yet another example of these enteric pathogens. It may be responsible for acute and at times overwhelming diarrhea-predominant gastrointestinal infection-like conditions. Indeed, many patients with chronic “diarrhea-predominant Irritable Bowel Syndrome” [D-IBS] may actually be chronically infected with Clostridium perfringens strains.

Perhaps the most clinically important gastrointestinal infection today is Clostridium difficile infection (CDI). CDI is caused by a gram-positive, spore-forming anaerobic bacillus with a terminal spore. CDI has led to an epidemic in North America with rapidly increasing incidence, severity of disease, and difficulty in treatment. It is the most common hospital acquired infection and may afflict as many as 3,000,000 patients in the US [McFarland et al., New Eng. J. Med., 320:204-10, 1989; Johnson et al., Lancet, 336:97-100, 1990].

Long term use of proton pump inhibitors and treatment of various conditions with antibiotics, and particularly with clindamycin and fluoroquinolones, appear to predispose patients to the development of Clostridium difficile infections.

CDI can result in asymptomatic colonization, mild loose motions or may progress to overwhelming severe diarrhea, pseudomembranous colitis, toxic megacolon, perforation, septicaemia and death. The recent epidemic of the NAP 1/027 strain of Clostridium difficile has resulted in a marked increase in morbidity and mortality in North America and Europe. This strain has caused clinically severe disease with markedly increased production of Toxin A and Toxin B, as well as the production of a third toxin, the binary toxin, and has been more resistant to antibiotics. Other important strains of CDI include the 017 and 014 epidemic strains. The CDI epidemic has reached new levels, with about 300 from an estimated 7178 patients present on any one day in U.S. hospitals expected to die as a result of the infections. [W Jarvis, et al. Am J Infection Control 2008; 37:263-270]. Therefore, there is an urgent need to develop life-saving and preventative therapies, preferably not relying on antibiotics, the most common root cause of CDI and many other enteric infections, for CDI and the other diseases related to enteric pathogens in animals, including humans.

### SUMMARY OF THE INVENTION

The present invention relates to compositions and methods for the treatment of enteric pathogen infections in animals, including humans, suffering from such infections or displaying diseases or conditions consistent with such infections.

In another embodiment, the invention relates to compositions and methods to prevent, or to at least reduce the likelihood of enteric pathogen infections in animals, including humans, that are at risk of such infections.

The invention in another embodiment relates to methods that comprise the serial or separated delivery of antibodies directed against enteric pathogens, to animals, including humans, suffering from infections related to such pathogens or displaying diseases or conditions consistent with such infections, or at risk of developing these infections, followed by the delivery to those subjects, of probiotics directed against at least some of such pathogens. These methods treat, prevent or reduce the acute and chronic infections and infestations of the gastrointestinal tract in humans and other animals by enteric pathogens.

In another embodiment, the invention relates to compositions that allow the serial or separated delivery to animals, including humans, suffering from infections caused by enteric pathogens or displaying diseases or conditions consistent with such infections, or at risk of developing such infections of antibodies directed against enteric pathogens and then the delivery of probiotics directed against the same pathogens.

In another embodiment, this invention relates to methods of making the compositions and using the methods of this invention.

In another embodiment, the invention relates to methods and compositions that treat, prevent or reduce clinical conditions or diseases which may be related to enteric pathogen infections but which causative pathogens are not known, e.g., IBS or travelers diarrhea. In spite of the absence of identifiable pathogens in such infections, the methods and compositions of this invention still have a positive effect on the various enteric pathogen infections driving these illnesses.

### DETAILED DESCRIPTION OF THE INVENTION

This invention relates to methods and compositions for the treatment and prophylaxis of enteric pathogen infections, diseases and conditions in animals, including humans.

The methods of the invention comprise the step of administering, serially or separately, (1) antibodies directed against a pathogen or group of pathogens that are related to enteric infections and (2) probiotics directed against at least some of said pathogen or group of pathogens to humans or other animals suffering from infections by the pathogens, or displaying diseases or conditions consistent with such infections, or at risk of developing such infections.

The compositions of the invention comprise antibodies directed against a pathogen or group of pathogens that are related to enteric infections and probiotics directed against at least some of those pathogens. The compositions of the invention are formulated or administered so as to prevent the antibodies and the probiotics coming into functional contact with each other before the antibodies have substantially bound to the pathogen(s) in the human or other animal.

As used herein “enteric pathogens” or pathogens that are “related to or consistent with enteric infections” refer to organisms capable of causing an infection in the gastrointestinal tract of an animal, including a human.

Examples of enteric pathogenic organisms include and are not limited to Aeromonas hydrophilia, Bacillus cereus, Vibrio parahemolyticus, Vibrio cholerae 01, Vibrio cholera non-01, Vibrio vulnificus, Salmonella enteric, Salmonella typhi, Salmonella paratyphi, Salmonella entertidis, Salmonella cholerasuis, Salmonella typhimurium, Clostridium difficile, Clostridium botulinum, Clostridium perfringens, Staphylococcus aureus, Escherichia coli (—various subclasses), Campylobacter jejuni, Campylobacter coli, Campylobacter lari, Campylobacter fetus, Yersinia enterocolitica, Yersinia pestis, Yersinia pseudotuberculosis, Plesiomonas shigelloides, Listeria monocytogenes, enteric viruses, for example, rotavirus, Norwalk-like viruses, enteric adenoviruses, coronavirus and all other non-enveloped enteroviruses, and enteric parasites and fungi, for example, Cryptosporidium, and Cyclospora, luminal and tissue mycobacteria, such as Mycobacterium avium avium, Mycobacterium avium paratuberculosis, Mycobacterium avium silvaticum and other components of the Mycobacterium avium complex, Helicobacter pylori, Giardia lamblia and other parasites including Dientamoeba fragilis, Blastocystis hominis and Entamoeba histolytica.

Among the diseases and infections, without limitation, that are related to or consistent with enteric pathogens are Irritable Bowel Syndrome in its various forms (diarrhea, pain, constipation, predominant or mixtures thereof), bloating, small bowel bacterial overgrowth, diverticular disease, including diverticulitis, colitis (ulcerative, Crohn's, lymphocytic, microscopic, indeterminate pseudo membranous, proctitis, post infective colitis) among others, as well as Crohn's Disease, idiopathic ileitis, constipation, flatulence, and halitosis, dysmotility conditions, including gastroparesis, reflux disease, pseudo-obstruction, bloating and traveler's diarrhea, as well as Parkinson's disease constipation.

As described herein, the invention in one embodiment comprises a pharmaceutical composition comprising antibodies directed against an enteric pathogen or group of enteric pathogens and a probiotic or group of probiotics that are likewise directed against at least some of those pathogens. As used herein, the composition may have the two components, the antibody component and the probiotic component, together in one delivery unit. In this embodiment, the two components are functionally separated in the delivery unit (e.g. by coating of capsules or microencapsulation). For example, the probiotic component may be encapsulated, such that it is delivered to the human or other animal, more slowly or later than the antibody component of the composition. Alternatively, the antibody component comprises the outside or outer layer of a capsule or other delivery unit or is coated on a capsule and the probiotic component comprises the inner part of the capsule. In either alternative, the probiotic component is delivered to the human or other animal after the antibody component has substantially bound to the pathogen(s).

A composition as used herein may also have the antibody component and the probiotic component in separate delivery systems or units. For example, two separate capsules, sachets, tablets, granules or pills. Again, the intent is to deliver the probiotic component after the antibody component has substantially bound to the pathogen(s).

The compositions of this invention can also comprise suppositories, enemas, or can be made into suspensions to be infused trans-endoscopically or trans-colonoscopically into the duodenum, terminal ileum or via an enteric tube into the jejunum. For example, in patients whose access to the GI tract has to be obtained in intensive care situations. The composition may also be administered combined with drinks or foods to be ingested serially, e.g., morning ingestion of the antibody component followed, preferably 4-12 hrs later, by the probiotic component of the composition.

The probiotic component of the composition of this invention is a micro organism selected, for example, from the group consisting of Lactobacilli, Bifidobacteria, E coli, Eubacteria, Saccharomyces species, Enterococci, Bacteroides or non pathogenic Clostridia, e.g. Clostridium butyricum and non-pathogenic C difficile. As will be appreciated by one of skilled in the art, other suitable probiotics known in the art may also be used in the compositions of the invention.

As used in this invention, the probiotic component is one or a group of probiotic organisms that are directed at the pathogen or group of pathogens being targeted or whose risk of infections is being reduced or prevented. As used herein, a probiotic(s) that is directed to an enteric pathogen or group of those pathogens are those probiotics that are capable in culture of eradicating or suppressing the growth of the targeted pathogen or pathogens. Thus, the probiotics of the compositions of this invention are preferably selected by co-culturing the enteric pathogens with a probiotic or groups thereof and selecting the probiotics or group that inhibits or suppresses growth of at least some of the pathogen(s).

The antibody component of the composition of this invention, may be polyclonal antibodies, monoclonal antibodies, Fab, Fab′, F(ab′).sub.2, Fv, dAb, and complementarity determining region (CDR) fragments, single-chain antibodies (scFv), chimeric antibodies, humanized or human antibodies, diabodies and polypeptides that contain at least a portion of an immunoglobulin that is sufficient to confer specific antigen binding. Preferably, they are polyclonal antibodies derived from eggs, including egg yolk and albumin, from poultry immunized with at least one antigen derived from at least one enteric pathogen. When the antibodies are monoclonal antibodies, they are also preferably mixtures of monoclonal antibodies or mixtures of monoclonal antibodies and polyclonal antibodies.

The antibodies of the compositions of this invention are directed against antigens of the enteric pathogens being targeted. The antigens may comprise whole organisms, spores, fimbriae, pilli capsules, glycocalyces, secreted enzymes, e.g. collagenase, hyaluronidase, coagulase, protease, immunoglobin, proteins isolated from cell membrane, lipopolysaccharide fraction as well as attenuated virus particles, toxins, viral proteins and cell surface proteins as well as fragments and mixtures thereof. Preferably, mixtures are used.

For example, it is particularly preferred to use as the antigen multiple strains of the pathogen(s) being targeted, or fragments or products of those strains. In one embodiment, for example, strains 027, 017, and 014 of CDI and fragments and toxins produced by those strains are used.

The antibody components of the compositions of the invention are preferably produced by immunizing a host, preferably poultry and most preferably chickens, with a diverse collection of antigens or immunogens or a group of antigens and immunogens derived from a group of pathogens. However, other hosts known in the art for antibody production including sheep, horses, and cows, can be used. Preferably, IgY polyclonal antibodies are used in the methods and compositions of the invention.

An alternate approach of antibody generation is the production of monoclonal antibodies. Again, however, it is preferred to use diverse mixtures of monoclonal antibodies in the methods and compositions of this invention. For example, when treating CDI, a mixture of monoclonal antibodies directed to Toxin A, Toxin B. or the binary toxin, or preferably all these are used. In the more preferred embodiments of a composition for treating CDI, the antibody component would contain a multiplicity of monoclonal antibodies, selected from the groups of antibodies directed to Toxin A, Toxin B, binary Toxin and supernatant toxins yet to be identified but able to be used to immunize from the supernantant, vegetative forms of the bacterium fimbriae, glycocalyces, pilli, spores, capsules, secreted enzymes e.g. collagenase, hyaluronidase, coagulase and immunoglobulin A protease, proteins and lipids isolated from the cell membranes and the lipopolysaccharide fraction spore and fractions of spores.

Monoclonal antibodies can be used alone (singularly) or preferably in combinations (mixtures) and alone or with polyclonal antibodies.

In a preferred embodiment when antibodies raised in chickens are used, the egg product, egg yolk alone or the whole egg content may be used to produce an antibody powder for administration after e.g. freeze drying spray drying. The whole egg versus yolk alone is preferred because of the increased amount of IgY available and the presence of the albumin as a “support” vehicle to stimulate the antibody-antigen binding within the GI tract during administration.

The amount of the antibody component and the probiotic component and the other treatment parameters of the compositions of this invention and in the methods of this invention are easily determined by those of skill in the art taking into account the patient, his or her history, the infection or condition being treated and the effect of various treatments.

The methods and compositions of the invention may also include additional steps or components. For example, in one embodiment, the human or other animal is pre-treated with antibiotics targeted against the enteric pathogens to reduce the infective load or numbers of the pathogens in the enteric tract. For example, in the situation where a patient is infected with Clostridium difficile pre-treatment with anti-clostridial agents including metronidazole, vancomycin, rifampicin, rifaximin, nitazoxanide or rifabutin used singly or in combinations, for at least one day and up to 3 months—reduces the load of the bacteria and spores.

In a further embodiment of the invention, the effectiveness of the methods and compositions may be enhanced by reducing the acid in the stomach during antibody ingestion to prevent acid damage of the antibody protein [curdling]. This can be achieved with H2 receptor antagonists, but more preferably by proton pump inhibitors—PPI's (e.g. omeprazole, lansoprazole, esomeprazole, pantoprazole, rabeprazole and other PPI's), administered prior to the ingestion of the medications. Ideally, the PPI should be given at least 2-4 hours before and in some situations a PPI could be combined with a H2RA to maximize acid suppression. This will allow the passage both of the probiotic component and especially the antibody component of the compositions of this invention through the gastric space without causing precipitation of the large proteins which make up the antibodies.

In some embodiments of the invention species other than humans benefit from methods compositions of this invention. In particular, for example, dogs which develop chronic Clostridium perfringens diarrhoea and other such diarrhea conditions caused by specific and non-specific pathogens may be treated in accordance with this invention.

In the prophylactic embodiment of this invention, the compositions of the invention are administered, often at lower doses than in the situation of active infection, to humans and other animals at risk of enteric pathogen infections.

### EXAMPLES

### Example 1

A 48 yr old female with longstanding and recurrent urine infections treated with antibiotics developed chronic diarrhoea. After a number of stool tests toxigenic Clostridium difficile was detected in the stool. This was a non-epidemic strain but nevertheless caused chronic diarrhoea which occurred between 10 and 15 times per day causing occasional incontinence.

The patient had been treated initially with 20 gm/d of C difficile immune egg powder preparation for 10 days but her stool continued to be C difficile-positive and her diarrhoea recurred. She was then given a combination of 10 gm of the same egg powder but this time together with Lactobacillus rhamnosus strain CDD1. This strain was selected because it could inhibit C. difficile in vitro and was added at a dose which was equivalent to 10¹⁰ bacteria for ten days. The antibodies were administered in the morning and the probiotic bacteria eight hours later for 10 days.

On completion of the study (at 4 and 8 weeks), the patient was free of C. difficile infection on stool tests. Her diarrhoea settled by day 3 of the combined therapy.

### Example 2

A 9 year old male allergic to penicillin was given prophylactic “clindamycin” following a cut finger which was then sutured in the emergency room in a San Francisco hospital. 3 to 4 weeks after finishing the clindamycin he developed diarrhoea. This diarrhoea was associated with cramping, urgency, malaise and progressive weight loss of about 2 to 3 kg. He was diagnosed as having the epidemic strain of C. difficile and was given metronidazole. He developed nausea and was then given vancomycin capsules 250 mg tds. His diarrhoea was inhibited quite effectively both by the metronidazole and by the vancomycin. Within 2 to 4 weeks of stopping the medications the diarrhoea would recur with up to 8 or 12 diarrhoeal stools per day. Over the next 18 months there were numerous recurrences of the diarrhoea each time suppressed by vancomycin. Numerous protocols of reducing doses of Vancomycin were tried but he continued to have diarrhoea.

The patient was then treated with 10 gm daily of the anti-C. difficile antibodies for 10 days. His diarrhoea initially settled but then recurred 3 to 4 weeks after the cessation of the 10 day treatment. He was then given a 10 gm morning dose of the antibody followed by an evening dose of a Bifidobacterium strain CDD2 strain that could inhibit C. difficile as a combination therapy for 10 days. The diarrhoea again ceased by day 3 and has not recurred now for nearly 8 months. His stool remain C difficile negative. The patient has gained weight, has formed stools, has no pain, no urgency and no incontinence.

1: A method for treating enteric pathogen infections in animals suffering from such infections or displaying diseases or conditions consistent with such infections, or for preventing or reducing the likelihood of enteric pathogen infections in animals at risk for developing such infections, the method comprising:
administering to said animal, serially or separately;
(1) an antibody or mixture of antibodies directed against said enteric pathogen(s); and
(2) a probiotic or mixture of probiotics directed against at least some of such enteric pathogen(s),
wherein the antibody component and the probiotic component are administered such that they are not in functional contact until the antibody component has substantially bound the enteric pathogen(s) in the animal.

2: A composition comprising:
(a) an antibody or mixture of antibodies directed against an enteric pathogen or group of said pathogens; and
(b) a probiotic or mixture of probiotics directed against at least some of said enteric pathogens or groups thereof, the antibody component not being in functional contact with the probiotic or mixture of probiotics.

3: The method of claim 1, wherein the animal is a human.

4: The method of claim 1, wherein the pathogen or group of pathogens are selected from the group consisting of Aeromonas hydrophilia, Bacillus cereus, Vibrio parahemolyticus, Vibrio cholerae 01, Vibrio cholera non-01, Vibrio vulnificus, Salmonella enteric, Salmonella typhi, Salmonella paratyphi, Salmonella entertidis, Salmonella cholerasuis, Salmonella typhimurium, Clostridium difficile, Clostridium botulinum, Clostridium perfringens, Staphylococcus aureus, Escherichia coli,
Campylobacter jejuni, Campylobacter coli, Campylobacter lari, Campylobacter fetus, Yersinia enterocolitica, Yersinia pestis, Yersinia pseudotuberculosis, Plesiomonas shigelloides, Listeria monocytogenes, enteric viruses, parasites and fungi, luminal and tissue mycobacteria, Helicobacter pylori, Giardia lamblia, Dientamoeba fragilis, Blastocystis hominis and Entamoeba histolytica.

5: The method of claim 4, wherein the enteric viruses are selected from the group consisting of rotavirus, Norwalk-like viruses, enteric adenoviruses, coronavirus and other non-enveloped enteroviruses,
wherein the fungi is selected from the group consisting of Cryptosporidium and Cyclospora,
and wherein the mycobacteria is selected from the group consisting of Mycobacterium avium avium, Mycobacterium avium paratuberculosis, and Mycobacterium avium silvaticum and other components of the Mycobacterium avium complex.

6: The method of claim 2, wherein the enteric pathogen or group of enteric pathogens is selected from the group consisting of enteric pathogens related to CDI and fragments, components, and products of those enteric pathogens.

7: The method of claim 2, wherein the antibody or group of antibodies are selected from the group consisting of polyclonal antibodies, monoclonal antibodies, mixtures of polyclonal antibodies and monoclonal antibodies, Fab, Fab′, F(ab′).sub.2, Fv, dAb, and complementarity determining region (CDR) fragments, single-chain antibodies (scFv), chimeric antibodies, humanized or human antibodies, diabodies and polypeptides that contain at least a portion of an immunoglobulin that is sufficient to confer specific antigen binding.

8: The method of claim 7, wherein the antibodies are selected from the group consisting of antibodies directed to Toxin A, Toxin B, binary Toxin and other supernatant toxins able to be used to immunize from the supernantant, vegetative forms of the bacterium fimbriae, glycocalyces, pilli, spores, capsules, secreted enzymes proteins and lipids isolated from the cell membranes and the lipopolysaccharide fraction spore and fractions of spores.

9: The method or the composition of claim 8, wherein the secreted enzymes are selected from the group consisting of collagenase, hyaluronidase, coagulase and immunoglobulin A protease.

10: The method of claim 1 wherein the antibody or group of antibodies are IgY antibodies raised in chickens.

11: The method of claim 1, wherein the probiotic or mixture of probiotics are selected from the group consisting of Lactobacilli, Bifidobacteria, E coli, Eubacteria, Saccharomyces, Enterococci, Bacteroides and non pathogenic Clostridia.

12: The method of claim 11, wherein the non pathogenic Clostridia is selected from the group consisting of Clostridium butyricum and non-pathogenic C difficile.

13: The method of claim 1, wherein the probiotic or mixture of probiotics are capable of eradicating or suppressing the growth of said pathogen(s) in vitro or in vivo.

14: The method of claim 1, wherein the infections are selected from the group consisting of Irritable Bowel Syndrome, bloating, small bowel bacterial overgrowth, diverticular disease, colitis, Crohn's Disease, idiopathic ileitis, constipation, flatulence, and halitosis, dysmotility conditions, reflux disease, pseudo-obstruction, bloating and traveler's diarrhea and Parkinson's disease constipation.

15: The method of claim 14, wherein the Irritable Bowel Syndrome is selected from the group consisting of diarrhea, pain, constipation, predominant and mixtures thereof,
wherein the diverticular disease is a diverticulitis,
wherein colitis is selected from the group consisting of ulcerative, Crohn's, lymphocytic, microscopic, indeterminate pseudo membranous, proctitis and post infective colitis,
and wherein the dysmotility condition is a gastroparesis.

16: The method of claim 1, wherein the antibody component and the probiotic component are contained in separate delivery systems or units.

17: The method of claim 16, wherein the delivery systems or units are in form of capsules, sachets, tablets, granules, pills, suppositories, enemas, or suspensions capable of being infused trans-endoscopically or trans-colonoscopically into the duodenum, terminal ileum or via an enteric tube into the jejunum.

18: The composition of claim 2, wherein the pathogen or group of pathogens are selected from the group consisting of Aeromonas hydrophilia, Bacillus cereus, Vibrio parahemolyticus, Vibrio cholerae 01, Vibrio cholera non-01, Vibrio vulnificus, Salmonella enteric, Salmonella typhi, Salmonella paratyphi, Salmonella entertidis, Salmonella cholerasuis, Salmonella typhimurium, Clostridium difficile, Clostridium botulinum, Clostridium perfringens, Staphylococcus aureus, Escherichia coli, Campylobacter jejuni, Campylobacter coli, Campylobacter lari, Campylobacter fetus, Yersinia enterocolitica, Yersinia pestis, Yersinia pseudotuberculosis, Plesiomonas shigelloides, Listeria monocytogenes, enteric viruses, parasites and fungi, luminal and tissue mycobacteria, Helicobacter pylori, Giardia lamblia, Dientamoeba fragilis, Blastocystis hominis and Entamoeba histolytica; or
the enteric viruses are selected from the group consisting of rotavirus, Norwalk-like viruses, enteric adenoviruses, coronavirus and other non-enveloped enteroviruses,
wherein the fungi is selected from the group consisting of Cryptosporidium and Cyclospora,
and wherein the mycobacteria is selected from the group consisting of Mycobacterium avium avium, Mycobacterium avium paratuberculosis, and Mycobacterium avium silvaticum and other components of the Mycobacterium avium complex, or
the enteric pathogen or group of enteric pathogens is selected from the group consisting of enteric pathogens related to CDI and fragments, components, and products of those enteric pathogens.

19: The composition of claim 2, wherein the antibody or group of antibodies are selected from the group consisting of polyclonal antibodies, monoclonal antibodies, mixtures of polyclonal antibodies and monoclonal antibodies, Fab, Fab′, F(ab′).sub.2, Fv, dAb, and complementarity determining region (CDR) fragments, single-chain antibodies (scFv), chimeric antibodies, humanized or human antibodies, diabodies and polypeptides that contain at least a portion of an immunoglobulin that is sufficient to confer specific antigen binding, or
wherein the antibodies are selected from the group consisting of antibodies directed to Toxin A, Toxin B, binary Toxin and other supernatant toxins able to be used to immunize from the supernantant, vegetative forms of the bacterium fimbriae, glycocalyces, pilli, spores, capsules, secreted enzymes proteins and lipids isolated from the cell membranes and the lipopolysaccharide fraction spore and fractions of spores.

20: The composition of claim 2 wherein the antibody component and the probiotic component are contained in separate delivery systems or units, or
wherein the composition is formulated with a delivery system or the composition is formulated as units in form of capsules, sachets, tablets, granules, pills, suppositories, enemas, or suspensions capable of being infused trans-endoscopically or trans-colonoscopically into the duodenum, terminal ileum, or via an enteric tube into the jejunum.
